(19) United States
(12) Patent Application Publication
(10) Pub. No.: US 2026/0027365 A1
(43) Pub. Date: Jan. 29, 2026

(54) Techniques For Providing Stimulus For Tinnitus Therapy

(71) Applicant: Cochlear Limited, Macquarie University, NSW (AU)

(72) Inventors: Remo Albert Gerardus Joseph Arts, Valkenswaards (NL); Kelly Assouly, Schaerbeek (BE)

(73) Assignee: Cochlear Limited, Macquarie University, NSW (AU)

(21) Appl. No.: 18/998,487

(22) PCT Filed: Jul. 22, 2023
(86) PCT No.: PCT/IB2023/057484

§ 371 Date: Jan. 25, 2025

Related U.S. Application Data
(60) Provisional application No. 63/393,215, filed on Jul. 28, 2022.

(51) Int. Cl. A61N 1/36; A61H 21/00; A61H 23/02

(52) U.S. Cl. CPC A61N 1/36038; A61H 21/00; A61H 23/0236; A61H 2201/5005; A61H 2205/027; A61H 2230/085

(57) ABSTRACT

A system includes an acoustic stimulator configured to provide an acoustic signal to an car of a recipient. The system also includes a measurement sensor configured to measure electrical potentials from the car of the recipient that are generated in response to the acoustic signal. The system further includes a tinnitus treatment stimulator configured to generate a stimulus to the car of the recipient based on the electrical potentials measured by the measurement sensor to provide tinnitus therapy to the recipient.

### CROSS REFERENCE TO RELATED APPLICATION

This patent application claims priority to U.S. provisional patent application 63/393,215, filed Jul. 28, 2022, which is incorporated by reference herein in its entirety.

### BACKGROUND

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### BRIEF SUMMARY

In one aspect disclosed herein, a method is provided that comprises measuring potentials from a recipient that are evoked in response to an acoustic signal provided to an ear of the recipient. The method further comprises providing stimulation, that is generated based on the potentials, to the ear of the recipient to treat tinnitus in the recipient.

In another aspect disclosed herein, a system is provided that comprises a measurement sensor configured to sense electrical potentials from an ear of a recipient that are generated in response to an acoustic signal. The system also comprises a tinnitus treatment stimulator configured to generate a stimulus to the ear of the recipient based on the electrical potentials sensed by the measurement sensor to provide tinnitus therapy to the recipient.

In still another aspect disclosed herein, a non-transitory computer-readable storage medium is provided that comprises computer-readable instructions stored thereon for causing a computer to perform an electrocochleography measurement from an ear of a recipient in response to acoustic stimulus provided to the ear, and to generate a signal for application to the ear based on the electrocochleography measurement to treat tinnitus in the recipient.

In still another aspect disclosed herein, a method provides therapy for tinnitus to a recipient. The method comprises recording electrical potential generated in an ear of the recipient in response to sound stimulation using electrocochleography. The method further comprises providing an electrical stimulus to the ear of the recipient that is generated based on the electrical potential.

### BRIEF DESCRIPTION OF THE DRAWINGS

FIG. 1A depicts a schematic diagram of an exemplary cochlear implant that can be configured to implement aspects of the techniques presented herein, according to some exemplary embodiments.

FIG. 1B depicts a block diagram of a system for treating tinnitus in a recipient of the cochlear implant of FIG. 1A, according to an embodiment.

FIG. 2A depicts a diagram of a system for treating tinnitus in a recipient, according to an embodiment.

FIG. 2B depicts a flow chart that illustrates examples of operations that can be performed to treat tinnitus in an ear of a recipient.

FIG. 3 depicts a table displaying examples of acoustic frequencies and electrodes that can be used in the operations of FIG. 2B to provide tinnitus treatment to a recipient.

FIG. 4 illustrates an example of a suitable computing system with which one or more of the disclosed embodiments can be implemented.

### DETAILED DESCRIPTION

Tinnitus is the perception of noise or “ringing” in the ears. Tinnitus is a common artefact of hearing loss, but tinnitus may also be a symptom of other underlying conditions, such as ear injuries, circulatory system disorders, etc. Tinnitus can, for example, be caused by abnormal hair cells in the cochlea of an ear generating erroneous signals. Although tinnitus effects can range from mild to severe, almost one-quarter of those individuals with tinnitus describe their tinnitus as disabling or nearly disabling. Tinnitus is an auditory phantom perception, which may be perceived as having various characteristics (e.g., pure tone; narrow band noise; polyphonic), and is experienced either unilaterally, bilaterally, or in the head. In some cases, the perception of tinnitus is intermittent or variable in magnitude.

Masking has been used to treat tinnitus, with either acoustic or electrical stimulation. Masking can comprise adding an audible or inaudible masking stimulus (e.g., signals) corresponding to sound (e.g., white noise; music; patterned sound; low-level sound; sound tailored based on characteristics of the recipient's tinnitus) intended to mask or cover up a phantom sound (e.g., ringing; hissing) caused by tinnitus. The added sound level can be close to, softer than, or louder than the perceived loudness of the phantom sound. While the tinnitus can be partially or fully masked by the added audible or inaudible sound such that the recipient's perception of the phantom sound is reduced, masking does not reduce or eliminate the tinnitus itself. In addition, some individuals may find the fitting procedure for a tinnitus device to be uncomfortable, particularly if prolonged conscious attention to one's tinnitus is required as the device is being adjusted.

According to some embodiments disclosed herein, systems and methods are provided for delivering an acoustic signal to an ear of a recipient experiencing tinnitus, measuring one or more potentials from the recipient that are evoked in response to the acoustic signal, and providing stimulation, that is generated based on the one or more potentials, to the ear of the recipient to treat the tinnitus in the recipient. The potentials can be measured, for example, by performing an electrocochleography (ECochG) measurement from the ear of the recipient using a measurement sensor after providing the acoustic signal to the ear. A tinnitus treatment stimulator, such as one or more electrodes can, for example, deliver an electrical stimulation to the ear based on the measured potential to mask tinnitus in the recipient, or in some cases, to reverse the pathophysiology of tinnitus. Alternatively, the stimulation provided to the ear of the recipient to treat the tinnitus can be an acoustic (or a combined electro-acoustic) stimulation. Acoustic stimulation can, for example, be provided to the ear of the recipient using an internal or external hearing aid device to provide tinnitus therapy.

Certain embodiments disclosed herein include objective techniques for providing guidance to a clinician on how to optimize the stimulation parameters to the ear of the recipient to treat tinnitus. Objective testing can also minimize the need for subjective testing, which is frequently associated with time consumption and other unwanted demands. Treatment of tinnitus can, for example, be achieved with stimulation representing environmental sounds, meaningless tinnitus-specific stimulation, or a combination of both. The stimulation can improve the tinnitus treatment effect when the stimulation is provided at one or more specific locations inside the ear of the recipient (e.g., in the cochlea or auditory nerve).

The eligibility of a recipient for tinnitus treatment can, for example, be tested using electrocochleography (ECochG). As used herein, electrocochleography (ECochG) refers to a technique of recording electrical potentials generated in the ear and/or auditory nerve of a recipient in response to acoustic stimulation. ECochG can also be used to diagnose tinnitus originating from the ear of a recipient. Tinnitus originating from a recipient's cochlea may, for example, be the most sensitive for extra-cochlear or intra-cochlear stimulation treatment. ECochG measurements can be performed on a recipient to assess the local neural health of the recipient and to reveal the optimal location for tinnitus treatment. The ECochG measurements can be analyzed to identify different potentials separately, and combinations of different potentials (i.e., evoked responses), that may be indicative of tinnitus characteristics in the recipient. Because ECochG provides objective measurements that do not require the participation of the recipient and that can be obtained in a relatively short amount of time, ECochG measurements can be used to assess tinnitus characteristics in a clinic.

ECochG measurements can, for example, be recorded inside the inner ear (i.e., inside the cochlea) or outside the inner ear of a recipient (e.g., the auditory nerve or elsewhere) of a recipient. The location of an ECochG measurement of a recipient can, for example, be selected based on the medical status of the recipient. For example, measurements can be recorded inside the inner ear in the case of an intracochlear cochlear implant recipient. Similarly, measurements can be recorded outside the inner ear in the case of a recipient of an extracochlear cochlear implant, or other implantable medical device located in the vicinity of the ear. Such other implantable medical devices can include vestibular stimulators, middle ear implants, auditory brainstem implants and the like. The ECochG measurements can provide insights (i.e., the prognostic value) to the effectiveness of electric and/or acoustic stimulation for tinnitus treatment. The ECochG measurements can also be used to determine the optimal stimulation parameters for tinnitus therapy, such as the location(s) (e.g., extra-cochlear or at various locations intra-cochlear) to stimulate the ear of a recipient to provide therapy for tinnitus. Testing for these stimulation parameters subjectively is a time consuming, demanding, and difficult process. ECochG can, for example, be used to identify which electrode in an array of electrodes (e.g., in a cochlear implant having an array of electrodes) is closest to a measured tinnitus frequency that can be targeted as a potential stimulation frequency or location for tinnitus therapy.

Certain embodiments disclosed herein can provide tinnitus treatment personalized to a recipient by providing stimulation to the ear of the recipient that is generated based on electrocochleography (ECochG) measurements (or other potential measurements) of the ear. ECochG can be used to help quickly and objectively determine the optimal location and other parameters for treating tinnitus in a recipient without the active participation of the recipient. ECochG can be used to predict the effectiveness of intra-cochlear electrical or acoustic stimulation to suppress the effect of tinnitus. ECochG can also be used to assess the possibility of effectively suppressing tinnitus with extra-cochlear electrical stimulation when the optimal location for stimulation can be reached with stimulation from outside the cochlea.

Electrocochleography (ECochG) testing is a clinical technique that can be used, for example, to assess the residual hearing of a recipient suffering from partial hearing loss. Electrocochleography testing can, for example, involve the delivery of acoustic stimuli to a recipient's ear, and then recording one or more responses of the ear (e.g., from residual functional hair cells) to the acoustic stimuli using an electrode in the ear canal, cochlea, or tympanic membrane. ECochG testing can be performed within a clinical environment, typically using equipment and techniques implemented by trained audiologists or clinicians. In particular, during ECochG testing procedures, a clinician plays predetermined clicks or tones to a recipient, while electrocochleography recordings are performed, for example, using an electrode in or near the recipient's middle ear or inner ear.

Merely for ease of description, the techniques presented herein are primarily described herein with reference to an illustrative medical device, namely a cochlear implant. However, it is to be appreciated that the techniques presented herein may also be used with a variety of other medical devices that, while providing a wide range of therapeutic benefits to recipients, patients, or other users, may benefit from the teachings herein used in other medical devices. For example, any techniques presented herein described for one type of hearing device, such as a cochlear implant, corresponds to a disclosure of another embodiment of using such teaching with another hearing device, including bone conduction devices (percutaneous, active transcutaneous and/or passive transcutaneous), middle ear auditory prostheses, direct acoustic stimulators, acoustic amplification hearing aids, consumer hearing devices, and also utilizing such with other electrically simulating auditory prostheses (e.g., auditory brain stimulators), etc.

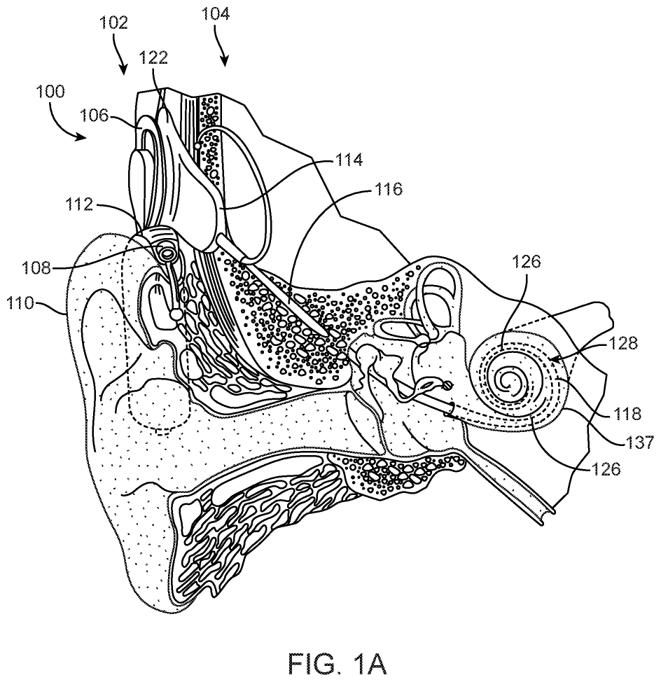

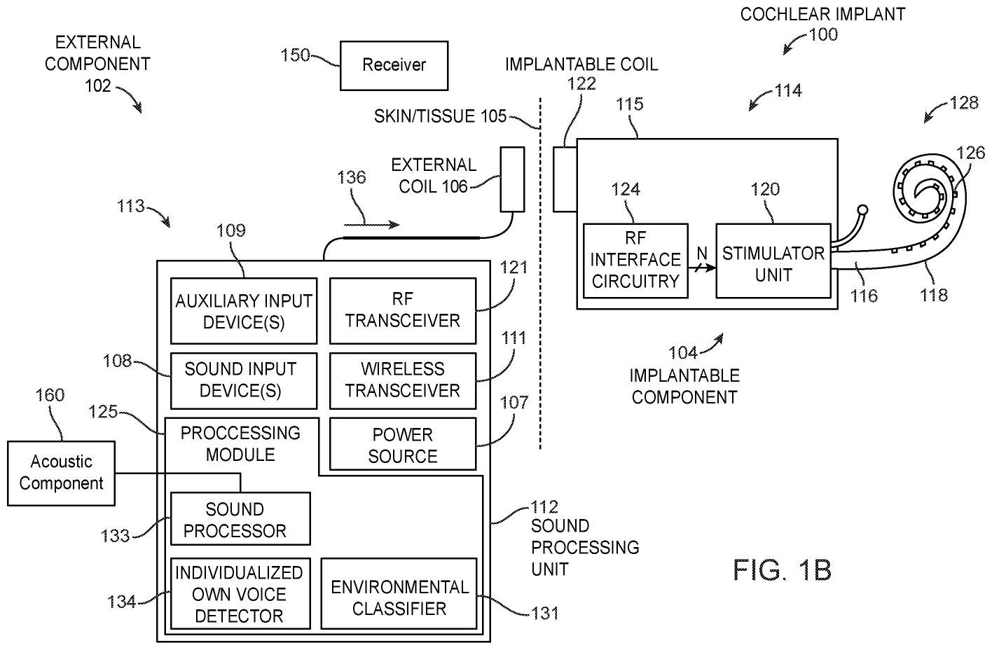

FIG. 1A is a schematic diagram of an exemplary cochlear implant 100 configured to implement aspects of the techniques presented herein. The cochlear implant 100 comprises an external component 102 and an internal/implantable component 104. The external component 102 is directly or indirectly attached to the body of the recipient and typically comprises an external coil 106 and, generally, a magnet (not shown in FIG. 1A) fixed relative to the external coil 106. The external component 102 also includes a sound processing unit 112. In the example of FIG. 1A, the sound processing unit 112 is a behind-the-ear (BTE) sound processing unit configured to be attached to, and worn behind, the auricle 110 of the recipient's ear. However, it is to be appreciated that sound processing unit 112 may have other arrangements, such as an off the ear (OTE) processing unit (e.g., a component having a generally cylindrical shape and that is configured to be magnetically coupled to the recipient's head), etc., a mini or micro-BTE unit, an in-the-canal unit that is configured to be located in the recipient's ear canal, a body-worn sound processing unit, etc.

In the exemplary embodiment of FIG. 1A, the implantable component 104 comprises an implant body (main module) 114, a lead region 116, and an intra-cochlear stimulating assembly 118, all configured to be implanted under the skin/tissue of the recipient. The implant body 114 includes an internal/implantable coil 122.

Stimulating assembly 118 is configured to be at least partially implanted in the recipient's cochlea 137. Stimulating assembly 118 includes a plurality of longitudinally spaced intra-cochlear electrical stimulating contacts (electrodes) 126 that collectively form a contact or electrode array 128 for delivery of electrical stimulation (current) to the recipient's cochlea 137. Stimulating assembly 118 extends through an opening in the recipient's cochlea (e.g., cochleostomy, the round window, etc.) and has a proximal end connected to a stimulator unit via lead region 116 and a hermetic feedthrough. Lead region 116 includes a plurality of conductors (wires) that electrically couple the electrodes 126 to the stimulator unit.

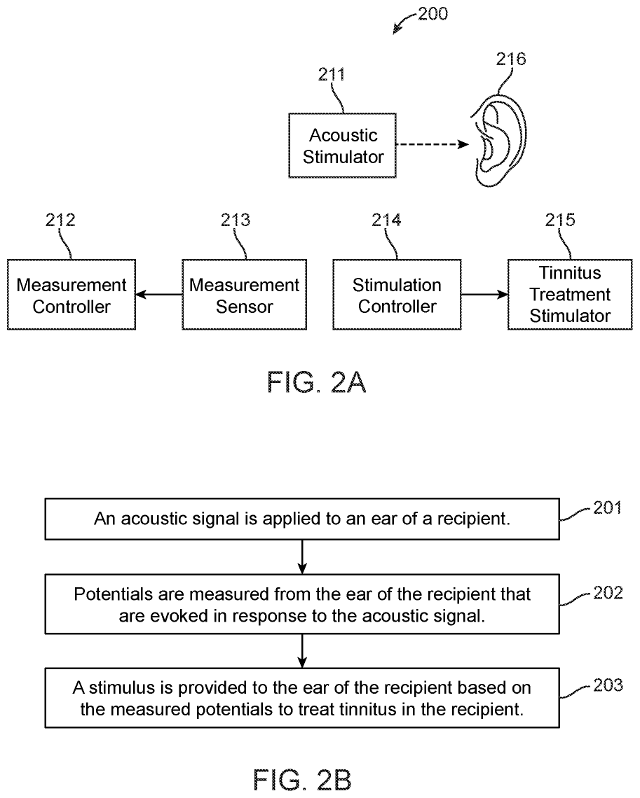

FIG. 1B is a diagram of a system for treating tinnitus in a recipient of the cochlear implant 100 of FIG. 1A, according to an embodiment. The system of FIG. 1B includes the cochlear implant 100 of FIG. 1A. The system of FIG. 1B also includes a receiver device 150 and/or an acoustic component 160. The acoustic component 160 is a sound generating device that generates one or more acoustic signals. Acoustic component 160 can, for example, be an acoustic component in an electrocochleography (ECochG) system. The acoustic component 160 can send one or more acoustic signals to the ear canal of the recipient via a sound tube. Acoustic component 160 can, for example, be coupled to, and controlled by, a sound processor 133 in sound processing unit 112 or by a computing system (not shown in FIG. 1B). The receiver 150 is another type of sound generating device that is located outside the ear of the recipient, such as a speaker or audio amplifier, or that is to be placed in the ear of the recipient, such as a hearing aid. Receiver 150 is used to generate one or more acoustic signals that are provided to the ear of the recipient. In some embodiments, the system of FIG. 1B may include only one of acoustic component 160 or receiver 150. The system of FIG. 1B can be used to treat tinnitus in a recipient, as disclosed in further detail below with respect to FIGS. 2A-2B.

FIG. 1B illustrates further details of the cochlear implant 100 of FIG. 1A. In the example of FIG. 1B, the external component 102 also comprises one or more input elements/devices 113 for receiving input signals at sound processing unit 112. In this example, the one or more input devices 113 include sound input devices 108 (e.g., microphones positioned by auricle 110 of the recipient, telecoils, etc.) configured to capture/receive input signals, one or more auxiliary input devices 109 (e.g., audio ports, such as a Direct Audio Input (DAI), data ports, such as a Universal Serial Bus (USB) port, cable port, etc.), and a wireless transmitter/receiver (transceiver) 111, each located in, on, or near the sound processing unit 112.

In the embodiment of FIG. 1B, the sound processing unit 112 also includes, for example, at least one power source 107, a radio-frequency (RF) transceiver 121, and a processing module 125. The processing module 125 comprises a number of elements, including an environmental classifier 131, the sound processor 133, and an individualized own voice detector 134. Each of the environmental classifier 131, the sound processor 133, and the individualized own voice detector 134 may be formed by one or more processors (e.g., one or more Digital Signal Processors (DSPs), one or more processing cores, etc.), firmware, software, etc. arranged to perform operations described herein. That is, the environmental classifier 131, the sound processor 133, and the individualized own voice detector 134 may each be implemented as firmware elements, partially or fully implemented with digital logic gates in one or more application-specific integrated circuits (ASICs), partially or fully in software, etc. The acoustic component 160 is connected to the sound processor 133.

As noted with respect to FIG. 1A, the cochlear implant 100 includes the external coil 106 and the implantable coil 122. The implantable coil 122 is part of the implant body 114, which is implanted under the skin/tissue 105 of the recipient. The coils 106 and 122 are typically wire antenna coils each comprised of multiple turns of electrically insulated single-strand or multi-strand wire. Generally, a magnet is fixed relative to each of the external coil 106 and the implantable coil 122. The magnets fixed relative to the external coil 106 and the implantable coil 122 facilitate the operational alignment of the external coil with the implantable coil 122. This operational alignment of the coils 106 and 122 enables the external component 102 to transmit data, as well as possibly power, to the implantable component 104 via a closely-coupled wireless link formed between the external coil 106 with the implantable coil 122. In certain examples, the closely-coupled wireless link is a radio frequency (RF) link. However, various other types of energy transfer, such as infrared (IR), electromagnetic, capacitive and inductive transfer, may be used to transfer the power and/or data from an external component to an implantable component and, as such, FIG. 1B illustrates only one exemplary arrangement.

As noted above, sound processing unit 112 of FIG. 1B includes the processing module 125. The processing module 125 is configured to convert input audio signals into stimulation control signals 136 for use in stimulating a first ear of a recipient (i.e., the processing module 125 is configured to perform sound processing on input audio signals received at the sound processing unit 112). Stated differently, the sound processor 133 (e.g., one or more processing elements implementing firmware, software, etc.) is configured to convert the captured input audio signals into stimulation control signals 136 that represent electrical stimulation for delivery to the recipient. The input audio signals that are processed and converted into stimulation control signals 136 may be audio signals received via the sound input devices 108, signals received via the auxiliary input devices 109, and/or signals received via the wireless transceiver 111.

In the embodiment of FIG. 1B, the stimulation control signals 136 are provided to the RF transceiver 121, which transcutaneously transfers the stimulation control signals 136 (e.g., in an encoded manner) to the implantable component 104 via external coil 106 and implantable coil 122. The stimulation control signals 136 are received at the RF interface circuitry 124 via implantable coil 122 and provided to the stimulator unit 120 in housing 115. The stimulator unit 120 is configured to utilize the stimulation control signals 136 to generate electrical stimulation signals (e.g., current signals) for delivery to the recipient's cochlea via lead 116 and one or more stimulating contacts 126 in electrode array 128. In this way, cochlear implant 100 electrically stimulates the recipient's auditory nerve cells, bypassing absent or defective hair cells that normally transduce acoustic vibrations into neural activity, in a manner that causes the recipient to perceive one or more components of the input audio signals.

FIG. 2A is a diagram of a system 200 for treating tinnitus in a recipient, according to an embodiment. The system 200 of FIG. 2A includes an acoustic stimulator 211, a measurement controller 212, a measurement sensor 213, a stimulation controller 214, and a tinnitus treatment stimulator 215. The acoustic stimulator 211 is a device that generates an acoustic signal for delivery to an ear 216 of a recipient experiencing tinnitus. The acoustic stimulator 211 can be, for example, the receiver 150 or the acoustic component 160 shown in FIG. 1B. The measurement sensor 213 measures one or more potentials from the ear 216 of the recipient that are evoked in response to the acoustic signal generated by the acoustic stimulator 211. The measurement sensor 213 can, for example, measure the potentials by performing an electrocochleography (ECochG) measurement from the ear 216 of the recipient after the acoustic signal is provided to the ear 216. The measurement sensor 213 is controlled by the measurement controller 212. The measurement controller 212 can receive signals from the measurement sensor 213 that are indicative of the potentials measured from the ear 216 of the recipient. The potentials measured from the ear 216 are processed by the measurement controller 212 (or another system) and are used to generate a stimulation for the ear 216 of the recipient. The tinnitus treatment stimulator 215 provides the stimulation, that is generated by the measurement controller 212 based on the one or more potentials measured by the measurement sensor 213, to the ear 216 of the recipient to treat the tinnitus in the recipient. The tinnitus treatment stimulator 215 is controlled by the stimulation controller 214. The stimulation controller 214 can provide control signals to the tinnitus treatment stimulator 215 for controlling the stimulation that the tinnitus treatment stimulator 215 provides to the ear 216 of the recipient to treat the tinnitus. Further details of the operation of the components of system 200 are described below with respect to FIG. 2B.

FIG. 2B depicts a flow chart that illustrates examples of operations that can be performed to treat tinnitus in an ear of a recipient. In operation 201, an acoustic signal (i.e., an acoustic stimulus) is applied to the ear of a recipient. The acoustic signal can be provided in operation 201 by the acoustic stimulator 211 of FIG. 2A, which can be, for example, the receiver 150 or the acoustic component 160 that are shown in FIG. 1B. In some embodiments, one or more acoustic stimulators 211, such as receiver 150 or acoustic component 160, can provide multiple acoustic signals to the ear of the recipient in operation 201. As an example that is not intended to be limiting, the acoustic stimulator 211 can be part of a hearing aid that is located in the recipient's ear or external to the recipient. The acoustic stimulator 211 can be an acoustic device, such as a speaker or audio amplifier, that is used to generate one or more acoustic signals in operation 201 to stimulate the ear of the recipient.

If the acoustic stimulator 211 in operation 201 is the acoustic component 160, the acoustic component 160 generates the one or more acoustic signals. The acoustic component 160 can be, for example, an acoustic component in an electrocochleography (ECochG) system. The acoustic component 160 sends one or more acoustic signals to the ear canal of the recipient via a sound tube in operation 201 in this example. Acoustic component 160 can, for example, be controlled by a computing system.

Alternatively, the cochlear implant 100 of FIGS. 1A-1B, including portions of the external component 102 and the internal component 104, such as stimulating contacts 126, can deliver one or more acoustic signals from the environment to the ear of the recipient in operation 201. One or more microphones in the sound processor 133 in the processing module 125 senses the one or more acoustic signals and sends the one or more acoustic signals to the sound processor 133. The sound processor 133 converts the one or more acoustic signals into a stimulation control signal 136 for use in stimulating the ear of a recipient by performing sound processing on the one or more acoustic signals. The stimulation control signal 136 is provided to the RF transceiver 121, which transfers the stimulation control signal 136 (e.g., in an encoded manner) to the implantable component 104 via external coil 106 and implantable coil 122. The RF interface circuitry 124 receives the stimulation control signal 136 via implantable coil 122 and provides the stimulation control signal 136 to the stimulator unit 120. The stimulator unit 120 is configured to utilize the stimulation control signal 136 to generate electrical stimulation signals (e.g., current signals) for delivery to the recipient's cochlea using the one or more stimulating contacts 126 (e.g., electrodes).

In operation 202, potentials are measured from the ear of the recipient that are evoked in response to the acoustic signal generated in operation 201. The potentials measured from the ear in operation 202 can be, for example, electrical potentials such as a voltage or a current. Operation 202 can, for example, be performed by the measurement sensor 213 of FIG. 2A. The measurement sensor 213 can measure the potentials from inside the ear (e.g., from the hair cells in the cochlea or from the auditory nerve) of the recipient or from outside the ear of the recipient in operation 202. Operation 202 can, for example, be performed as an electrocochleography (ECochG) measurement that uses the measurement sensor 213 to measure electrical potentials generated from inside or outside the ear of the recipient in response to the acoustic signal generated in operation 201. The measurement sensor 213 can measure two or more potentials (e.g., electrical potentials) from the ear of the recipient in operation 202. The potentials can be isolated, for example, from the ECochG measurement using processing circuitry (e.g., in measurement controller 212).

As an example that is not intended to be limiting, the measurement sensor 213 can include one or more electrodes that measure the potentials from the ear of the recipient. The electrodes can, for example, be used to implement the ECochG measurement. In these embodiments, the electrodes in the measurement sensor 213 of operation 202 can be, for example, invasive electrodes, such as electrodes in transtympanic (TT) needles, or non-invasive electrodes, such as extratympanic (ET) electrodes. Invasive electrodes typically generate clearer and more robust electrical responses (e.g., with larger amplitudes) to the acoustic signal, because invasive electrodes are close to the voltage generators in the ear of the recipient. A TT needle can, for example, be placed on the promontory wall of the middle ear and the round window. Non-invasive electrodes have the advantage of not causing pain or discomfort to the recipient. The use of non-invasive electrodes does not require the sedation, anesthesia, or medical supervision of the recipient. However, non-invasive electrodes typically generate responses to the acoustic signal that are smaller in magnitude.

As other examples, the measurement sensor 213 used in operation 202 can include one or more of the stimulating contacts 126 (e.g., electrodes) of the cochlear implant 100 of FIGS. 1A-1B, as described above, or the stimulating contacts of any other type of ear implant. In this example, the stimulating contacts 126 can be used to measure electrical potentials in the cochlea of the recipient (e.g., from hair cells) that are evoked in response to the acoustic signal(s) generated in operation 201. The electrical potentials measured by the stimulating contacts 126 are delivered as one or more signals through lead region 116 to the RF interface circuitry 124 or other receiving circuitry in housing 115. The signals indicative of the measured electrical potentials are then transmitted from the receiving circuitry in housing 115 to the implantable coil 122. The implantable coil 122 then transmits the signals indicative of the measured electrical potentials through a wire and/or wirelessly to the RF transceiver 121 via external coil 106. The RF transceiver 121 then transmits the signals indicative of the electrical potentials measured from the ear to the processing module 125 for processing and analysis or externally. The measurement controller 212 can include sound processing unit 112 or be a separate device in this embodiment.

A clinician or a computer system can then determine optimal stimulation parameters to apply to the ear of the recipient based on the potentials measured in operation 202 to treat tinnitus. As an example, the clinician or computer system can determine the optimal stimulation parameters to apply to the ear of the recipient based on an electrocochleography (ECochG) measurement of the ear of the recipient performed in operation 202. The potentials measured by the measurement sensor 213 in operation 202 can be used to determine the optimal stimulation parameters, such as the location of the cause of the tinnitus, the location of potential tinnitus treatment, and the parameters of the stimulus to be applied to the recipient. The potentials measured by the measurement sensor 213 in operation 202 can also be used to determine the potential effectiveness of stimulation for tinnitus treatment. The potentials can be analyzed separately and in different combinations to determine the location of the tinnitus and/or the location of potential tinnitus treatment. The location of the cause of the tinnitus and/or the location of the potential tinnitus treatment can, for example, correspond to one or more stimulating contacts that are inside or outside the ear of the recipient, such as the various electrodes discussed above.

In operation 203, a stimulus is provided to the ear of the recipient based on the potentials measured in operation 202 to treat tinnitus in the recipient. Operation 203 can, for example, be performed by the tinnitus treatment stimulator 215 of FIG. 2A. If the measurement sensor 213 measures the electrical potentials in operation 202 as an electrocochleography (ECochG) measurement, then the stimulus can be provided to the ear of the recipient in operation 203 based on the ECochG measurement to treat tinnitus in the recipient. An advantage of using an ECochG measurement is that ECochG can help to locate an optimal and individualized location for applying electrical stimulation to ameliorate perceptions of tinnitus. Further, using objective measures, such as ECochG, in programming stimulation controller 214 and/or tinnitus treatment device 215 can advantageously minimize programming time and/or recipient distress that may be caused by the recipient focusing on their tinnitus during programming.

The tinnitus treatment stimulator 215 can, for example, provide a masking stimulus to the ear of the recipient in operation 203 that masks or reduces a phantom sound (e.g., ringing or hissing) caused by tinnitus. The masking stimulus can, for example, include white noise, music, patterned sound, low-level sound, calming sound, or sound that has been tailored based on the characteristics of the recipient's tinnitus determined using the potentials measured in operation 202. The masking stimulus can, for example, include a tone that is different than the frequency of the tinnitus to train the recipient's brain not to focus on the tinnitus. Instead of, or in addition to, providing a masking stimulus, a stimulus can be provided to the ear of the recipient in operation 203 that reverses the pathophysiology of a phantom sound causing the tinnitus. The sound level added by the stimulus in operation 203 can be close to, louder than, or quieter than the perceived loudness of the phantom sound.

The potentials measured in operation 202 can be used to determine the parameters of the stimulus applied to the ear of the recipient in operation 203. For example, potentials measured by the measurement sensor 213 in operation 202 can be used to determine the shape and/or size of one or more waveforms that comprise the stimulus applied to the ear of the recipient in operation 203. As specific examples, the potentials measured in operation 202 can be used to determine the amplitude of the stimulus, the pulse rate of the stimulus, the frequency of the stimulus, the duty cycle of the stimulus, or other characteristics of the stimulus applied in operation 203.

The stimulus provided to the ear of the recipient in operation 203 can be, for example, an electrical stimulus (e.g., a current or voltage signal), an acoustic stimulus (i.e., one or more sound signals), or a combined electro-acoustic stimulus for providing tinnitus treatment to the recipient. If the tinnitus treatment stimulator 215 generates an acoustic stimulus in operation 203, the tinnitus treatment stimulator 215 can, for example, be an acoustic device, such as a speaker, acoustic actuator, or audio amplifier in a hearing aid device or in another type of device. If the tinnitus treatment stimulator 215 generates an electrical stimulus in operation 203, the tinnitus treatment stimulator 215 can, for example, include one or more electrodes that provide the stimulus to the ear of the recipient. The electrodes in the tinnitus treatment stimulator 215 can be invasive electrodes or non-invasive electrodes, such as ET electrodes.

In an embodiment, the tinnitus treatment stimulator 215 in operation 203 can include one or more of the stimulating contacts 126 (e.g., electrodes) of the cochlear implant 100 of FIGS. 1A-1B disclosed herein. In this embodiment, the processing module 125 can convert an input stimulus generated based on the potentials measured in operation 202 into the stimulation control signal 136. The input stimulus can be received at the sound processing unit 112 from acoustic component 160, sound input devices 108, auxiliary input devices 109, and/or wireless transceiver 111 (or generated internally) and then provided to processing module 125. The stimulation control signal 136 is provided to the RF transceiver 121. RF transceiver 121 transfers the stimulation control signal 136 to the implantable component 104 via external coil 106 and implantable coil 122. The stimulation control signal 136 is provided to the RF interface circuitry 124 via implantable coil 122. Then, the stimulation control signal 136 is provided to the stimulator unit 120. The stimulator unit 120 utilizes the stimulation control signal 136 to generate electrical stimulation signals (e.g., current signals) for delivery to the recipient's cochlea via the one or more stimulating contacts 126 (e.g., electrodes). The electrical stimulation signals delivered to the recipient's cochlea in this embodiment can, for example, include the input stimulus (provided in operation 203) superimposed with the input audio signals described above with respect to FIGS. 1A-1B.

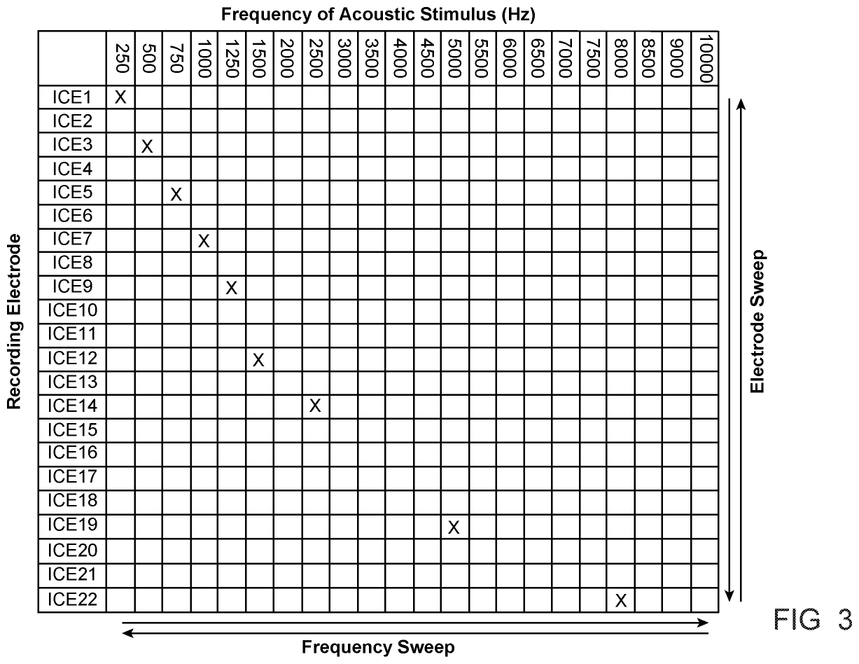

In embodiments that use multiple electrodes positioned at different locations to deliver an electrical stimulation to the ear of the recipient in operation 203, such as the stimulating contacts 126 in cochlear implant 100, the stimulus can be provided by electrically stimulating one or more of the electrodes at selected locations using selected frequencies and/or selected amplitudes to treat tinnitus. An example of one of these embodiments is disclosed herein with respect to FIG. 3. FIG. 3 depicts a table displaying examples of acoustic frequencies and recording electrodes that can be used in the operations of FIG. 2B to provide a personalized tinnitus treatment to a recipient. FIG. 3 displays examples of 22 frequencies of acoustic stimulus ranging from 250 hertz (Hz) to 10,000 Hz that can be provided to the ear of a recipient by acoustic stimulator 211 in operation 201, such as receiver 150 or acoustic component 160.

In the example of FIG. 3, the measurement sensor 213 of operation 202 includes up to 22 recording electrodes that are identified as electrodes ICE1-ICE22 in the table of FIG. 3. The electrodes ICE1-ICE22 of FIG. 3 are configured to measure electrical potentials from the ear of the recipient that are evoked in response to the acoustic signals generated by acoustic stimulator 211 in operation 201. For example, the electrodes ICE1-ICE22 are configured to measure electrical potentials in the ear of the recipient that are evoked by acoustic frequencies in the range of 250-10,000 Hz generated by the acoustic stimulator 211. The X marks in FIG. 3 indicate the center frequencies of the filter band for 9 of the electrodes ICE1, ICE3, ICE5, ICE7, ICE9, ICE12, ICE14, ICE19, and ICE22 using an exemplary sound processing strategy. In an exemplary embodiment, the 22 electrodes ICE1-ICE22 shown in the table of FIG. 3 are intra-cochlear electrodes, such as the stimulating contacts 126 in cochlear implant 100. In some embodiments, the electrical potentials that are measured by 1, 2 or all 3 of the electrodes ICE20, ICE21, and ICE22 in the table of FIG. 3 in response to acoustic frequencies can also be measured by one or more extra-cochlear electrodes (e.g., electrodes located outside the recipient's cochlea or ear).

In some embodiments, one or more acoustic stimulators 211 generate the acoustic frequencies shown in FIG. 3 (or a subset of these frequencies) in operation 201 at different times during an ascending frequency sweep in the direction shown by the arrow pointing right at the bottom of FIG. 3. In other embodiments, one or more acoustic stimulators 211 generate the acoustic frequencies shown in FIG. 3 (or a subset of these frequencies) in operation 201 at different times during a descending frequency sweep in the direction shown by the arrow pointing left at the bottom of FIG. 3. In operation 202, one or more of the electrodes ICE1-ICE22 measure the electrical potentials generated in the ear of the recipient (e.g., in the cochlea or auditory nerve of the recipient) in response to the acoustic frequencies generated in operation 201. The electrodes ICE1-ICE22 can, for example, sense the electrical potentials in an ascending frequency electrode sweep from ICE1 to ICE22, as shown by the down pointing arrow in FIG. 3, or in a descending frequency electrode sweep from ICE22 to ICE1, as shown by the up pointing arrow in FIG. 3. The electrodes ICE1-ICE22 can, for example, measure the electrical potentials using ECochG measurements.

By using the electrode and frequency sweeps shown in the example of FIG. 3, several electrical potentials can be measured to find the deviant location(s) or frequencies in the ear of the recipient that cause tinnitus. The electrodes ICE1-ICE22 that sense electrical potentials that indicate deviant location(s) or frequencies can be selected to provide electrical stimulus to the ear of the recipient in operation 203 to treat tinnitus in the recipient. These techniques can be used to provide a personalized test to find the optimal stimulation parameters (e.g., the location, stimulus frequency, or stimulus amplitude) for intra-cochlear and/or extra-cochlear electrical or acoustic stimulation to treat tinnitus. As examples, if absent or enlarged electrical potentials or abnormal ratios between different electrical potentials are measured by one or more of the electrodes ICE1-ICE22 in operation 202, then these electrodes (e.g., having a range of frequencies) can be selected for electrical stimulation to treat tinnitus in the recipient in operation 203. The potential measurements and the tinnitus treatment can advantageously be provided without participation of the recipient.

In an embodiment, the components shown in FIG. 2A can be part of an on-board system that is worn by, and/or implanted in, the recipient. The stimulation controller 214 shown in FIG. 2A can include a program that generates the control signals indicative of the stimulation to be provided for the tinnitus treatment in operation 203 by the tinnitus treatment stimulator 215. The on-board system can be reconfigured, whereby the recipient can initiate an ECochG testing function in the on-board system, and the program in the stimulation controller 214 that generates the control signals for the tinnitus stimulation can be automatically updated according to the latest ECochG test results.

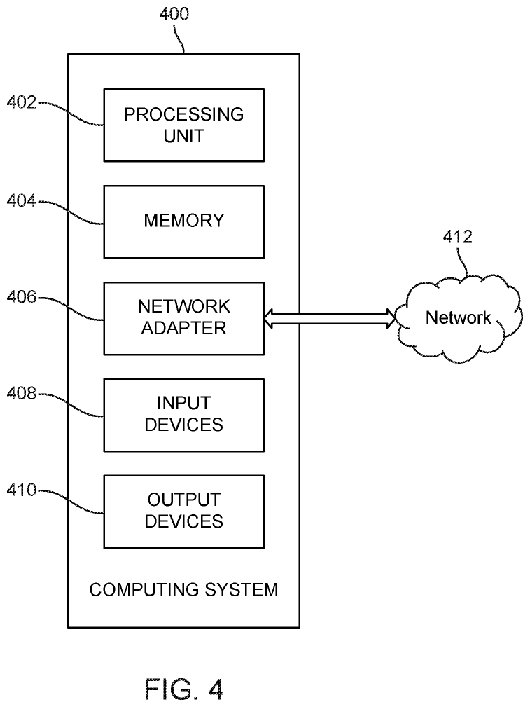

FIG. 4 illustrates an example of a suitable computing system 400 with which one or more of the disclosed examples can be implemented. For example, computing system 400 can be used to determine the optimal stimulation parameters to apply to the ear of the recipient in operation 203 to treat tinnitus based on the potentials measured by the measurement sensor 213 in operation 202. Computing systems, environments, or configurations that can be suitable for use with examples described herein include, but are not limited to, personal computers, server computers, hand-held devices, laptop devices, multiprocessor systems, microprocessor-based systems, programmable consumer electronics (e.g., smart phones), network computers, minicomputers, mainframe computers, tablets, distributed computing environments that include any of the above systems or devices, and the like. The computing system 400 can be a single virtual or physical device operating in a networked environment over communication links to one or more remote devices. The remote device can be an auditory prosthesis (e.g., the auditory prosthesis of FIGS. 1A-1B), a personal computer, a server, a router, a network personal computer, a peer device or other common network node.

Computing system 400 includes at least one processing unit 402 and memory 404. The processing unit 402 includes one or more hardware or software processors (e.g., Central Processing Units) that can obtain and execute instructions. The processing unit 402 can communicate with and control the performance of other components of the computing system 400. The memory 404 is one or more software-based or hardware-based computer-readable storage media operable to store information accessible by the processing unit 402.

The memory 404 can store instructions executable by the processing unit 402 to implement applications or cause performance of operations described herein, as well as store other data. The memory 404 can be volatile memory (e.g., random access memory or RAM), non-volatile memory (e.g., read-only memory or ROM), or combinations thereof. The memory 404 can include transitory memory or non-transitory memory. The memory 404 can also include one or more removable or non-removable storage devices. In examples, the memory 404 can include non-transitory computer-readable media, such as RAM, ROM, EEPROM (Electronically-Erasable Programmable Read-Only Memory), flash memory, optical disc storage, magnetic storage, solid state storage, or any other memory media usable to store information for later access. In examples, the memory 404 encompasses a modulated data signal (e.g., a signal that has one or more of its characteristics set or changed in such a manner as to encode information in the signal), such as a carrier wave or other transport mechanism and includes any information delivery media. By way of example, and not limitation, the memory 404 can include wired media such as a wired network or direct-wired connection, and wireless media such as acoustic, radio-frequency, infrared and other wireless media or combinations thereof.

In the illustrated example, the system 400 further includes a network adapter 406, one or more input devices 408, and one or more output devices 410. The system 400 can include other components, such as a system bus, component interfaces, a graphics system, a power source (e.g., a battery), among other components.

The network adapter 406 is a component of the computing system 400 that provides network access to network 412. The network adapter 406 can provide wired or wireless network access and can support one or more of a variety of communication technologies and protocols, such as ETHERNET, cellular, BLUETOOTH, near-field communication, and RF (Radiofrequency), among others. The network adapter 406 can include one or more antennas and associated components configured for wireless communication according to one or more wireless communication technologies and protocols.

The one or more input devices 408 are devices over which the computing system 400 receives input from a user. The one or more input devices 408 can include physically-actuatable user-interface elements (e.g., buttons, switches, or dials), touch screens, keyboards, mice, pens, and voice input devices, among others input devices.

The one or more output devices 410 are devices by which the computing system 400 is able to provide output to a user. The output devices 410 can include, displays, speakers, and printers, among other output devices.
Additional examples are now described. Example 1 is a method comprising: measuring potentials from a recipient that are evoked in response to an acoustic signal provided to an ear of the recipient; and providing stimulation, that is generated based on the potentials, to the ear of the recipient to treat tinnitus in the recipient.
In Example 2, the method of Example 1 can further include, wherein measuring the potentials from the recipient comprises measuring electrical potentials from the recipient using at least one electrode.
In Example 3, the method of any one of Examples 1-2 can further include, wherein measuring the potentials from the recipient comprises performing electrocochleography using a measurement sensor to measure the potentials from the recipient that are evoked in response to the acoustic signal.
In Example 4, the method of any one of Examples 1-3 can further include, wherein providing the stimulation to the ear of the recipient to treat the tinnitus comprises providing electrical stimulation to the ear of the recipient using an electrode.
In Example 5, the method of any one of Examples 1-4 can further include, wherein providing the stimulation to the ear of the recipient to treat the tinnitus comprises providing an acoustic stimulation to the ear of the recipient using an acoustic device.
In Example 6, the method of any one of Examples 1-5 further comprises: determining a location to provide the stimulation and a frequency of the stimulation based on the potentials measured from the recipient.
In Example 7, the method of Example 6 can further include, wherein providing the stimulation to the ear of the recipient to treat the tinnitus comprises providing the stimulation at the location and at the frequency determined based on the potentials measured from the recipient.
Example 8 is a system comprising: a measurement sensor configured to sense electrical potentials from an ear of a recipient that are generated in response to an acoustic signal; and a tinnitus treatment stimulator configured to provide a stimulus to the ear of the recipient based on the electrical potentials sensed by the measurement sensor to provide tinnitus therapy to the recipient.
In Example 9, the system of Example 8 further comprises: an acoustic stimulator configured to provide the acoustic signal to the ear of the recipient.
In Example 10, the system of Example 9 can further include, wherein the acoustic stimulator comprises an acoustic component in an electrocochleography system that is configured to generate the acoustic signal.
In Example 11, the system of any one of Examples 8-10 can further include, wherein the tinnitus treatment stimulator is configured to provide the stimulus to the ear of the recipient at a location or at a frequency determined based on the electrical potentials sensed by the measurement sensor.
In Example 12, the system of any one of Examples 8-11 can further include, wherein the measurement sensor is configured to measure the electrical potentials during electrocochleography.
In Example 13, the system of any one of Examples 8-12 can further include, wherein at least one of the measurement sensor or the tinnitus treatment stimulator comprises at least one electrode.
In Example 14, the system of Example 13 can further include, wherein the at least one electrode is configured to provide the stimulus to the ear of the recipient as at least one electrical signal with a waveform characteristic that is selected based on the electrical potentials.
In Example 15, the system of Example 14 can further include, wherein the at least one electrode is configured to provide the stimulus to the ear of the recipient as the at least one electrical signal having a frequency selected based on the electrical potentials.
In Example 16, the system of any one of Examples 8-15 can further include, wherein the tinnitus treatment stimulator comprises an array of electrodes positioned at different locations with respect to the recipient, and wherein the tinnitus treatment stimulator is configured to provide the stimulus to the ear of the recipient as at least one electrical signal provided to one or more of the electrodes that are selected based on the electrical potentials.
In Example 17, the system of any one of Examples 8-16 can further include, a measurement controller that processes signals received from the measurement sensor, and wherein the tinnitus treatment stimulator is controlled by a stimulation controller.
Example 18 is a non-transitory computer-readable storage medium comprising computer-readable instructions stored thereon for causing a computer to: perform an electrocochleography measurement from an ear of a recipient in response to acoustic stimulus provided to the ear; and generate a signal for application to the ear based on the electrocochleography measurement to treat tinnitus in the recipient.
In Example 19, the non-transitory computer-readable storage medium of Example 18 can further include, wherein the computer-readable instructions further cause the computer to: provide the acoustic stimulus to the ear of the recipient.
In Example 20, the non-transitory computer-readable storage medium of any one of Examples 18-19 can further include, wherein the computer-readable instructions further cause the computer to: generate the signal for application to the ear based on the electrocochleography measurement indicating at least one abnormal electrical potential measured from the ear of the recipient.
In Example 21, the non-transitory computer-readable storage medium of any one of Examples 18-20 can further include, wherein the computer-readable instructions further cause the computer to: generate signals having multiple frequencies for application to the ear of the recipient based on the electrocochleography measurement to treat the tinnitus in the recipient.
In Example 22, the non-transitory computer-readable storage medium of any one of Examples 18-21 can further include, wherein the computer-readable instructions further cause the computer to: generate signals for application to multiple locations in the ear of the recipient based on the electrocochleography measurement to treat the tinnitus in the recipient.
Example 23 is a method for providing therapy for tinnitus to a recipient, wherein the method comprises: recording an electrical potential generated in an ear of the recipient in response to sound stimulation; and providing an electrical stimulus to the ear of the recipient that is generated based on the electrical potential.
In Example 24, the method of Example 23 further comprises: generating the sound stimulation in the ear of the recipient.
In Example 25, the method of any one of Examples 23-24 can further include, wherein recording the electrical potential generated in the ear of the recipient comprises: recording electrical potentials generated in the ear of the recipient in response to multiple acoustic frequencies in the sound stimulation.
In Example 26, the method of any one of Examples 23-25 can further include, wherein providing the electrical stimulus to the ear of the recipient comprises: providing a masking stimulus to the ear that masks a phantom sound caused by the tinnitus.
In Example 27, the method of any one of Examples 23-26 can further include, wherein providing the electrical stimulus to the ear of the recipient comprises: providing electrical signals to the ear that have a range of frequencies that are selected based on the electrical potential.
In Example 28, the method of any one of Examples 23-27 can further include, wherein recording the electrical potential generated in the ear of the recipient comprises: recording the electrical potential generated in the ear of the recipient using electrocochleography.
In Example 29, the method of any one of Examples 23-28 can further include, wherein providing the electrical stimulus to the ear of the recipient comprises: reversing pathophysiology causing the tinnitus.

Any embodiment or any feature disclosed herein can be combined with any one or more other embodiments and/or other features disclosed herein, unless explicitly indicated otherwise. Any embodiment or any feature disclosed herein can be explicitly excluded from use with any one or more other embodiments and/or other features disclosed herein, unless explicitly indicated otherwise. It is noted that any method detailed herein also corresponds to a disclosure of a device and/or system configured to execute one or more or all of the method actions associated with the device and/or system as detailed herein. It is further noted that any disclosure of a device and/or system detailed herein corresponds to a method of making and/or using that device and/or system, including a method of using that device according to the functionality detailed herein.

The foregoing description of the exemplary embodiments of the present invention has been presented for the purpose of illustration. The foregoing description is not intended to be exhaustive or to limit the present invention to the examples disclosed herein. In some instances, features of the present invention can be employed without a corresponding use of other features as set forth. Many modifications, substitutions, and variations are possible in light of the above teachings, without departing from the scope of the present invention.

1. A method comprising:
measuring potentials from a recipient that are evoked in response to an acoustic signal provided to an ear of the recipient; and
providing stimulation, that is generated based on the potentials, to the ear of the recipient to treat tinnitus in the recipient.

2. The method of claim 1, wherein measuring the potentials from the recipient comprises measuring electrical potentials from the recipient using at least one electrode.

3. The method of claim 1, wherein measuring the potentials from the recipient comprises performing electrocochleography using a measurement sensor to measure the potentials from the recipient that are evoked in response to the acoustic signal.

4. The method of claim 1, wherein providing the stimulation to the ear of the recipient to treat the tinnitus comprises providing electrical stimulation to the ear of the recipient using an electrode.

5. The method of claim 1, wherein providing the stimulation to the ear of the recipient to treat the tinnitus comprises providing an acoustic stimulation to the ear of the recipient using an acoustic device.

6. The method of claim 1 further comprising:
determining a location to provide the stimulation and a frequency of the stimulation based on the potentials measured from the recipient.

7. The method of claim 6, wherein providing the stimulation to the ear of the recipient to treat the tinnitus comprises providing the stimulation at the location and at the frequency determined based on the potentials measured from the recipient.

8. A system comprising:
a measurement sensor configured to measure electrical potentials from an ear of a recipient that are generated in response to an acoustic signal; and
a tinnitus treatment stimulator configured to provide a stimulus to the ear of the recipient based on the electrical potentials measured by the measurement sensor to provide tinnitus therapy to the recipient.

9. The system of claim 8 further comprising:
an acoustic stimulator configured to provide the acoustic signal to the ear of the recipient.

10. The system of claim 9, wherein the acoustic stimulator comprises an acoustic component in an electrocochleography system that is configured to generate the acoustic signal.

11. The system of claim 8, wherein the tinnitus treatment stimulator is configured to provide the stimulus to the ear of the recipient at a location or at a frequency determined based on the electrical potentials measured by the measurement sensor.

12. The system of claim 8, wherein the measurement sensor is configured to measure the electrical potentials during electrocochleography.

13. The system of claim 8, wherein at least one of the measurement sensor or the tinnitus treatment stimulator comprises at least one electrode.

14. The system of claim 13, wherein the at least one electrode is configured to provide the stimulus to the ear of the recipient as at least one electrical signal with a waveform characteristic that is selected based on the electrical potentials.

15. The system of claim 14, wherein the at least one electrode is configured to provide the stimulus to the ear of the recipient as the at least one electrical signal having a frequency selected based on the electrical potentials.

16. The system of claim 8, wherein the tinnitus treatment stimulator comprises an array of electrodes positioned at different locations with respect to the recipient, and wherein the tinnitus treatment stimulator is configured to provide the stimulus to the ear of the recipient as at least one electrical signal provided to one or more of the electrodes that are selected based on the electrical potentials.

17. (canceled)

18. A non-transitory computer-readable storage medium comprising computer-readable instructions stored thereon for causing a computer to:
perform an electrocochleography measurement from an ear of a recipient in response to acoustic stimulus provided to the ear; and
generate a signal for application to the ear based on the electrocochleography measurement to treat tinnitus in the recipient.

19. The non-transitory computer-readable storage medium of claim 18, wherein the computer-readable instructions further cause the computer to:
provide the acoustic stimulus to the ear of the recipient.

20. The non-transitory computer-readable storage medium of claim 18, wherein the computer-readable instructions further cause the computer to:
generate the signal for application to the ear based on the electrocochleography measurement indicating at least one abnormal electrical potential measured from the ear of the recipient.

21. The non-transitory computer-readable storage medium of claim 18, wherein the computer-readable instructions further cause the computer to:
generate signals having multiple frequencies for application to the ear of the recipient based on the electrocochleography measurement to treat the tinnitus in the recipient.

22. (canceled)

23. (canceled)

24. (canceled)

25. (canceled)

26. (canceled)

27. (canceled)

28. (canceled)

29. (canceled)
